(19) United States
(12) Patent Application Publication
(10) Pub. No.: US 2016/0303051 A1
(43) Pub. Date: Oct. 20, 2016

(54) Burst Drug Release Compositions

(71) Applicant: Wyeth LLC, New York, NY (US)

(72) Inventors: Kevin Scott Kinter, Glen Allen, VA (US); Peter J. Ramsey, Midlothian, VA (US)

(73) Assignee: Wyeth LLC, New York, NY (US)

(21) Appl. No.: 15/133,977

(22) Filed: Apr. 20, 2016

Related U.S. Application Data
(63) Continuation of application No. 13/662,079, filed on Oct. 26, 2012.
Application No. 13/662,079 is a continuation-in-part of application No. 12/779,130, filed on May 13, 2010, now abandoned.
(60) Provisional application No. 61/177,943, filed on May 13, 2009.

(51) Int. Cl. A61K 9/20; A61K 31/192; A61K 9/24

(52) U.S. Cl. CPC A61K 9/2095; A61K 9/209; A61K 31/192; A61K 9/2054

(57) ABSTRACT

Solid dose compositions comprising at least one low solubility pharmaceutically active ingredient, at least one low solubility filler and at least one controlled release agent. comprising at least one poorly soluble pharmaceutically active ingredient, a insoluble binder and at least one controlled release agent along with a method of manufacturing said composition are disclosed. Methods for making these compositions are also disclosed.

### BACKGROUND

Burst drug release from extended release hydrophilic matrix tablets is an evolving area of pharmaceutics. The pharmaceutical industry employs various methods for compounding pharmaceutical agents in tablet formulations. In addition to active ingredients, formulations include other excipients such as controlled release agents, diluents, binders, disintegrants, surface active agents, glidants, lubricants, colorants, coating substances, surfactants and many other raw materials that impart different properties to the final solid dosage product.

Further, certain processing steps are utilized to formulate and accurately formulate and/or manufacture solid dose products. The most common processing steps associated with preparing solid dose formulations are summarized below:

“Wet Granulation” methods can be used where the flow properties of a compound such as an active pharmaceutical ingredient (“API”) are poor which can result in poor content uniformity when formulated as a dry blend. Wet granulation is commonly used to improve the processing characteristics of a powder blend, including improved flowability, content uniformity and more uniform particle size.

Wet granulation can be used to improve flow, compressibility, bio-availability, homogeneity, electrostatic properties, and stability of solid dosage forms. Granulation is often required to improve the flow of powder mixtures and mechanical properties of tablets. Granules are usually obtained by adding liquids (binder or solvent solutions). Larger quantities of granulating liquid produce a narrower particle size range and coarser and harder granules, i.e. the proportion of fine granulate particles decreases. The particle size of the granulate is determined, in part, by the quantity and addition rate of granulating fluid.

Wet granulation methods can be used where the flow properties of a compound such as an active pharmaceutical ingredient (“API”) are poor which may result in poor content uniformity when formulated as a dry blend. Wet granulation is commonly used to improve the processing characteristics of a powder blend, including improved flowability, compaction properties, content uniformity and more uniform particle size. The use of water and heat in wet granulation may cause chemical degradation or physical form conversion.

Process variables encountered in the formation of the granules can lead to significant tableting challenges. Granule properties can be affected by viscosity of the granulating solution, the rate of addition of granulating solution, the type of mixer used and duration of mixing, method and rate of dry and wet blending. The above variables can change the density and the particle size of the resulting granules and may have a major influence on fill weight and compaction qualities. The wet granulation drying operation can also result Soluble API migration to the surface of the granules may also occur during the drying operation.

“Direct Compression” is defined as the process by which tablets are compressed directly from powder mixture of API and suitable excipients. No pretreatment of the powder blend by wet or dry granulation procedure is required. It involves only blending and compression. This offers the advantage of faster production because it requires fewer unit operations, less machinery, and generally less processing time along with, in some cases, increased product stability. In case of directly compressed tablets, after disintegration, each primary drug particle is liberated.

While having all the benefits a granulation process can provide such as improving material flow behavior and content uniformity, “Roller Compaction,”, a dry granulation technique, offers advantages over wet granulation for moisture, solvent or heat sensitive compounds.

In roller compaction, powder is fed to two counter-rotating rolls which draw the powder between the rolls due to friction, which compacts the powder. Roller compaction is seemingly a simple process but the fundamental mechanisms are complex due to a significant number of material properties and machine variables such as material flow properties, friction against roll surface, compressibility, compactibility, elastic properties, air permeability, roll surface, roll dimension, roll pressure, roll gap, roll speed, feed method and conditions.

Although there are a large variety of processing factors and parameters that can be manipulated, there are generally three controllable parameters in the roller compaction process: roll pressure, roll gap and roll speed. Because the consolidation of a powder blend into ribbons is the result of mechanical stress (normal and shear stresses) within the powder during roller compaction, all the parameters are studied by examining their correlation to the normal (compressive) stress and the shear stress.

The viscosity of rate controlling polymers is another material property that is relevant to solid dosage pharmaceutical compositions, though viscosity is most commonly recognized as a property which characterizes the flow nature of a liquid. In the pharmaceutical arts, viscosity becomes relevant with respect to solid dosage forms such as tablets and capsules once these are taken orally and are exposed to the fluids in the digestive tract including the mouth, throat, stomach and intestines.

Controlled release agents are commonly included as excipients in pharmaceutical formulations. Such extended release agents, preferably a substituted cellulose derivative, such as hydroxypropylmethyl cellulose (HPMC) facilitate the extended release of the pharmaceutically active ingredients from the formulation such that the formulation can be administered to a patient less often, such as once daily. It is preferably present in an amount that allows for the formation of a gel matrix from which the active ingredient is gradually released. In addition, compositions contemplated herein may comprise further extended release agents, preferably those that swell upon contact with water such as polyvinylpyrrolidone, hydroxyethylcellulose, hydroxypropylcellulose, other cellulose ethers and esters like methylcellulose, methylethylcellulose, hydroxypropylmethylcellulose, carboxymethylcellulose, starch, pregelatinized starch, polymethacrylate, polyvinylacetate, microcrystalline cellulose, dextrans or mixtures thereof.

### BRIEF DESCRIPTION OF DRAWINGS

Features illustrated in the Figures are not drawn to scale unless explicitly stated otherwise, and the relative sizes of certain features may be exaggerated to better illustrate the features. Embodiments will be described with reference to the following figures, in which like numerals represent like items throughout the figures, and in which:

FIG. 1 Summarizes results comparing API release and composition apparent viscosity in compositions processed with wet granulation, roller compaction and direct compression processes.

FIG. 2 Summarizes a composition comparison using pre-mix batch A.

FIG. 3 Summarizes a composition comparison using pre-mix batch B.

FIG. 4 Summarizes a composition comparison using pre-mix batch C.

FIG. 5 Summarizes a composition comparison using pre-mix batch D.

FIG. 6 Summarizes a composition comparison using pre-mix batch E.

FIG. 7 Summarizes a composition comparison using pre-mix batch F.

FIG. 8 Summarizes a composition comparison using pre-mix batch G.

FIG. 9 Summarizes a composition comparison using pre-mix batch H.

FIG. 10 Shows the differences in the release profile of ibuprofen in an ER formulation when utilizing a soluble binder versus an insoluble binder when the formulation was prepared using roller compaction.

FIG. 11 Shows the differences in the release profile of ibuprofen in an ER formulation when utilizing a soluble binder versus an insoluble binder when the formulation was prepared using wet granulation.

### SUMMARY OF THE INVENTION

The inventors have found that certain kinds of processing on ingredients in formulations that included pharmaceutical actives and controlled release agents with certain apparent viscosities have marked effect on the dissolution of the active ingredients. In the pharmaceutical sciences, controlling the dissolution rate of active ingredients can be critical to the desired release timing and functionality of the active ingredient(s). As such, the discoveries and compositions disclosed herein offer novel approaches to controlling the dissolution of active ingredients through unique combinations of ingredients and using specifically processed active ingredient(s) in the compositions.

### DESCRIPTION OF THE INVENTION AND EXAMPLES

One specific observation and advantage of the inventions disclosed herein is that the process of wet granulation of the mix of pharmaceutically active ingredients plus controlled release agents gave surprising results in solid formulations with respect to the burst characteristics of pharmaceutically active ingredients at certain viscosities. Specifically, higher viscosity formulations where the mix of pharmaceutically active ingredients plus controlled release agents were processed using wet granulation had consistently faster burst rates.

Conversely, another specific observation and advantage of the inventions disclosed herein is that the processes of direct compression and roller compaction showed similar characteristics to each other with respect to burst characteristics of pharmaceutically active ingredients at certain viscosities. Specifically, higher viscosity formulations where the mix of pharmaceutically active ingredients plus controlled release agents were processed using direct compression and roller compaction had consistently lower burst rates than those processed using wet granulation.

The invention will allow the formulation of pharmaceuticals wherein release profiles can be adjusted to create compositions with both Immediate Release (IR) and Extended Release (ER) characteristics. As a preferred embodiment, with respect to pain management products, a critical need is to have an initial dose released up front to provide analgesia along with efficacious blood levels for extended time periods. Compositions, exemplified herein, offer such advantageous characteristics.

Further to the inventors' conception with respect to an IR/ER formulation, bilayer formulations provide advantages in certain circumstances. From a dissolution perspective, drug release can be optimized by keeping the layer formulations with different burst characteristics separated. It may also be advantageous to separate the IR layer from the ER layer to allow the IR portion to fully dissolve quickly in the GI tract prior to the gel matrix formation that extends the release and delays absorption of the ER component. Another aspect of potential compositions embodied by this invention is that biphasic drug release patterns can be achieved, as with a bilayer IR/ER formulation. Such formulations can be created with a monolithic design. This is a bit contrary to commonly used multilayer approaches to IR/ER formulations, but monolithic prototypes can have significant advantages from a manufacturing perspective. The tablet/capsule press can be run faster as the compression events that are occurring are less complicated. Additionally, with tablet presses that are double sided, production rates can be doubled. Also, tablet specifications are more straight forward as you are only dealing with a single layer. As such, there is less concern with the layer adhesion.

With regard to active ingredients, the preferred embodiment includes NSAIDs present in an analgesia-inducing or pain-alleviating amounts. Of the cyclooxygenase-1 inhibitors useful in the practice of the present invention, including those that are mentioned as being preferred, ibuprofen may be present in the claimed compositions in amounts ranging from about 50 to about 800 mg. Preferably it is present in amounts ranging from about 200 to about 600 mg. Most preferably, it is present in an amount of about 600 mg. The terms “effective amount” or “therapeutically effective amount” of an active agent as provided herein is defined as an amount of the agent at least sufficient to provide the desired therapeutic effect. The exact amount required will vary from subject to subject, depending on age, general condition of the subject, the severity of the condition being treated, and the particular active agent administered, and the like.

The term “normal approved dose” of an active agent as provided herein is defined as an amount of the agent that has been approved as safe and effective by the United States Food and Drug Administration for administration in humans in a particular dosage form. An approved dose is thus a dose found in a pharmaceutical product, an amount of active agent per unit dosage form. In the present invention, reference to a ratio of approved doses means doses approved for the same patient population (e.g., adult to adult or pediatric to pediatric), and approved for the same dosage form (e.g., elixir, tablet, capsule, caplet, controlled release, etc.).

In the practice of the invention, one of ordinary skill in the art can take an approved dosage form of any over-the-counter (OTC) or prescription decongestant and/or antihistamine, reduce it by, e.g., 25% to 50% or more, and co-administer it with an approved amount (dose) of a NSAID to achieve effective relief of rhinitis with reduced side effects.

The present invention contemplates compositions comprising either a single or multiple pharmaceutically active ingredients (i.e., decongestant, antihistamine and NSAID).

The non-steroidal anti-inflammatory drugs (NSAID's) for use in the pharmaceutical compositions and methods of use of the present invention may be selected from any of the following categories:

(1) The propionic acid derivatives;

(2) the acetic acid derivatives;

(3) The fenamic acid derivatives

(4) The biphenylcarboxylic acid derivatives;

(5) The oxicams, and

(6) Cox-2 inhibitors

Accordingly, the term “NSAID” as used herein is intended to mean any non-steroidal anti-inflammatory compound, including the pharmaceutically acceptable non-toxic salts thereof, falling within one of the six structural categories above.

The specific compounds falling within the foregoing definition of the non-steroidal anti-inflammatory drugs for use in the present invention are well known to those skilled in the art and reference may be found in various literature reference sources for their chemical structures, pharmacological activities, side effects, normal dosage ranges, etc. See, for example, Physician's Desk Reference, and The Merck Index.

Of the propionic acid derivatives for use herein, ibuprofen, naxproxen, flurbiprofen, fenoprofen, ketoprofen, suprofen, fenbufen, and fluprofen are specifically contemplated. Of the acetic acid derivatives, exemplary compounds include tolmetin sodium, zomepirac, sulindac and indomethacin. Of the fenamic acid derivatives, exemplary compounds include mefenamic acid and meclofenamate sodium. Exemplary biphenylcarboxlic acid derivatives for use in the present invention include diflunisal and flufenisal. Exemplary oxicams include piroxicam, sudoxicam and isoxicam. Exemplary Cox-2 inhibitors include celecoxib, rofecoxib, meloxicam, and nimesulide. Of the foregoing non-steroidal anti-inflammatory drugs, in the practice of the exemplified embodiments of the present invention, ibuprofen is exemplified.

With respect to the dosage amount of the non-steroidal anti-inflammatory drugs in the compositions of the invention, although the specific dose will vary depending upon the age and weight of the patient, the severity of the symptoms, the incidence of side effects and the like, for humans, typical effective analgesic amounts of NSAID's are about 200-1000 mg diflunisal, about 50-200 mg zomepirac sodium, about 100-800 mg ibuprofen, more preferably 600 mg ibuprofen, about 250-1000 mg naproxen, about 50-200 mg flurbiprofen, about 100-400 mg fenoprogen, about 20-40 mg piroxicam, about 250-500 mg mefanamic acid, about 200-800 mg fenbufen or about 50-100 mg ketoprofen; however, greater or lesser amounts may be employed if desired or necessary.

The term “antihistamine”, used in connection with treating nasal symptoms associated with allergy or cold, generally refers to histamine H₁ receptor antagonists. Numerous chemical substances are known to have histamine H₁ receptor antagonist activity. Many useful compounds can be classified as ethanolamines, ethylenediamines, alkylamines, phenothiazines or piperidines. Representative H₁ receptor antagonists, include, without limitation: astemizole, azatadine, azelastine, acrivastine, brompheniramine, chlorpheniramine, clemastine, cyclizine, carebastine, cyproheptadine, carbinoxamine, descarboethoxyloratadine (also known as SCH-34117), desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine and triprolidine. Other compounds can readily be evaluated to determine activity at H₁ receptors by known methods, including specific blockade of the contractile response to histamine of isolated guinea pig ileum.

Chlorpheniramine is specifically contemplated herein. The usual adult dosage of chlorpheniramine is 4 mg orally every 4-6 hours as needed, up to a maximum of 24 mg per day. The usual pediatric dosage of chlorpheniramine is 2 mg orally every 4-6 hours, up to a maximum of 12 mg per day. The preferred salt is chlorpheniramine maleate. In accordance with the present invention, the usual adult dosage is thus reduced to 3 mg, or further to 2 mg, orally every 4-6 hours as needed, up to a maximum of 12-18 mg per day. Similarly, in an embodiment of the invention, the pediatric dosage is 1.5 mg, or 1 mg, orally every 4-6 hours, up to a maximum of 6-9 mg per day. In a further embodiment, the invention permits combining a pediatric dosage of chlorpheniramine with an adult dosage of an NSAID, such as ibuprofen.

The decongestants for use in the pharmaceutical compositions and methods of use of the present invention include, but are not limited to, pseudoephedrine, phenylephedrine, phenylpropanolamine. One of skill in the art would know of many other appropriate decongestants and their approved dosages.

Pseudoephedrine and phenylephedrine are specifically contemplated herein. The usual adult dose of pseudoephedrine is 60 mg every 4-6 hours, up to a maximum of 240 mg per day. The usual pediatric dose of pseudoephedrine is 15 mg every 6 hours, up to a maximum of 60 mg per day for ages 2-5 and 30 mg every 6 hours, up to a maximum of 120 mg per day for ages 6-12. Thus, in specific embodiments of the practice of the present invention, the adult dose can be reduced to 45 or 30 mg every 4-6 hours, with a maximum of 120 to 180 mg per day, and the pediatric dose can be reduced to about 11 or 7.5 mg every 6 hours, up to a maximum of 30-45 mg per day. From the foregoing it is apparent that the invention contemplates administering a double pediatric dose with a normal adult dose of an NSAID to an adult.

Anti-tussives act on the brain to suppress the cough reflex. Such cough suppressants are used to relieve dry persistent coughs. The most commonly used drugs are dextromethorphan (an NMDA receptor antagonist), codeine and pholcodine (which are opioids. However, one skilled in the art would understand that there are many other well known and common anti-tussives that may be used. The present invention is optionally direct to the use of anti-tussives. The anti-tussive may be used in amounts of less than or equal to 75% of the approved dosage.

With respect to the solubility of the contemplated APIs, it is generally known that a drug substance is considered highly soluble when the highest dose strength is soluble in <250 ml water over a pH range of 1 to 7.5. Measures for quantifying solubility of both APIs and excipients are also generally known. The Biopharmaceutics Classification System (“BCS”), which is used by the FDA, is well known and accepted and incorporated herein by reference. The BCS has 4 different classes, I, II, III, and IV, as well as criteria for determining whether an ingredient is of high permeability, high solubility, low permeability or low solubility. As an example, Ibuprofen is considered a BCS II drug, which is high permeability and low solubility. Similar parameters for measurement of excipients are also applicable and contemplated. With respect to excipients, some examples of generally recognized water insoluble Fillers are Starches (Corn, Potato, Wheat, Rice, Pea), modified Starches and partially Pregelatinized Starches, Powdered Cellulose, Microcrystalline Cellulose (MCC), Silicified MCC, Calcium Carbonates and others. Commonly recognized Soluble Fillers include Sugars (Fructose, Dextrose, Sucrose, Maltose, Lactose), Sugar Derivatives (Dextrates, Maltodextrin) and Sugar Alcohols (Mannitol, Sorbitol, Erythritol, Isomalt, Xylitol) and others.

Compositions of the invention are formulated in a solid single dosage form such as tablets, capsules, and the like. Solid compounds will typically be administered orally.

Exemplary compositions of the present invention are directed to solid dosage forms such as bulk powders, tablets, caplets, pellets, capsules, granules, and any other dosage form suitable for oral administration. For purposes of this specification and the accompanying claims, the term “tablet” refers equally to a tablet, a caplet or any other solid dosage form which is suitable for oral administration.

Also contemplated are the inclusion of one or more non-pharmaceutically active excipients in the compositions of the present invention. These include, but are not limited to, controlled release agents, diluents, binders, disintegrants, surface active agents, glidants, lubricants, colorants, coating substances, surfactants and many other raw materials that impart different properties to the final solid dosage product.

Controlled release agents are commonly included as excipients in pharmaceutical formulations. Such extended release agents, preferably a substituted cellulose derivative, such as hydroxypropylmethyl cellulose (HPMC) facilitate the delayed release of the pharmaceutically active ingredients from the formulation such that the formulation can be administered to a patient less often, such as once daily, as compared to immediate release formulations of the same API(s). It is preferably present in an amount that allows for the formation of a gel matrix from which the active ingredient is gradually released. In addition, composition contemplated herein may comprise further extended release agents, preferably those that swell upon contact with water such as polyvinylpyrrolidone, hydroxyethylcellulose, hydroxypropylcellulose, other cellulose ethers and esters like methylcellulose, methylethylcellulose, hydroxypropylmethylcellulose, carboxymethylcellulose, starch, pregelatinized starch, polymethacrylate, polyvinylacetate, microcrystalline cellulose, dextrans or mixtures thereof. Generally, controlled release agents are present in an amount from about 0.5% to about 50% of the weight of the final composition and more specifically from about 10% to about 30% of the weight of the final composition.

Binders are agents used to impart cohesive qualities to the powdered material. Binders impart cohesiveness to the tablet formulation which ensures the tablet integrity following compression, as well as improving the free-flowing qualities by the formulation of granules of desired hardness and size. Suitable binder materials include, but are not limited to, starch (including corn starch and pregelatinzed starch), gelatin, sugars (including sucrose, glucose, dextrose, lactose and sorbitol), polyethylene glycol, waxes, natural and synthetic gums, e.g., acacia, tragacanth, sodium alginate, celluloses such as Microcrystalline Cellulose and synthetic polymers such as polymethacrylates and polyvinylpyrrolidone.

With respect to the formulation of the burst profile ER layer, as disclosed in examples, the inventors surprisingly found that utilizing mostly insoluble filler/binder created an advantageous formulation with respect to the release profile of an insoluble active ingredient, such as ibuprofen. This is contrary to the common wisdom with respect to formulation science, where insoluble active ingredients are generally paired with soluble binders. For example, Dextrates is commonly used as a filler/binder that is characterized as ideal for chewable and soluble tablets. Brands of Dextrates, such as EMDEX®, are known and marketed as appropriate to deliver the required flow, compaction, taste masking and flavor carrying capacity. It is highly water-soluble and gives a cool smooth mouth feel. Dextrates is a purified mixture of saccharides resulting from the controlled enzymatic hydrolysis of starch.

In contrast, the inventors exemplified formulations utilizing an essentially water insoluble fillers/binders/diluents, e.g. microcrystalline cellulose (MCC), which is a non-caloric bulking agent, anti-caking agent, and emulsifier. Cellulose is a compound derived from high quality wood pulp. MCC is essentially insoluble in water and does not gel like methylcellulose. It also provides natural source of dietary fiber. That said, one of skill in the formulation arts would not use an insoluble filler/binder with an insoluble API, but rather would try a soluble filler/binder. Other known insoluble filers include starches (corn, potato, wheat, rice, etc.), modified starches and pregelatinized starches, powdered cellulose, calcium carbonate and calcium phosphates.

Lubricants have a number of functions in tablet manufacture. They prevent adhesion of the tablet material to the surface of the dies and punches, reduce interparticle friction, facilitate the ejection of the tablets from the die cavity and may improve the rate of flow of the tablet granulation. Examples of suitable lubricants include, but are not limited to, magnesium stearate, calcium stearate, stearic acid, glyceryl behenate, talc, sodium lauryl sulfate, sodium stearyl fumarate, polyethylene glycol or mixtures thereof. Generally, the lubricant is present in an amount from about 0.25% to about 5% of the weight of the final composition and more specifically from about 0.5 to about 1.5% of the weight of the final composition.

A disintegrant is a substance, or a mixture of substances, added to a tablet to facilitate its breakup or disintegration after administration. Materials serving as disintegrants have been classified chemically as starches, clay, celluloses, alginates, gums and cross-linked polymers. Examples of suitable disintegrants include, but are not limited to, crosscarmelose sodium, sodium starch glycolate, starch, magnesium aluminum silicate, colloidal silicon dioxide, methylcellulose, agar, bentonite, alginic acid, guar gum, citrus pulp, carboxymethyl cellulose, microcrystalline cellulose, or mixtures thereof. Generally, the disintegrant is present in an amount from 0% to about 30% of the weight of the final composition and more specifically from about 0% to about 15% of the weight of the final composition.

Glidants are substances which improve the flow characteristics of a powder mixture. Examples of glidants include, but are not limited to colloidal silicon dioxide, talc or mixtures thereof. Generally, the glidant is present in an amount of from about 0.1% to about 10% of the weight of the final composition and more specifically from 5 about 0.1% to about 5% of the weight of the final composition.

The adsorbent may be, for example colloidal silicon dioxide, microcrystalline cellulose, calcium silicate or mixtures thereof. Generally, the adsorbent is present in an amount from about 0.05% to about 42% of the weight of the final composition and more specifically from about 0.05% to about 37% of the weight of the final composition.

If desired, other ingredients, such as diluents, stabilizers and anti-adherents, conventionally used for pharmaceutical formulations may be included in the present formulations. Optional ingredients include coloring and flavoring agents which are well known in the art.

The pharmaceutical composition described in the present invention may be formulated to release the active ingredients in an extended release manner. Various formulations are contemplated for dosage forms of these components.

The invention is further described by means of the following examples, which are not intended to limit the scope of the claimed invention in any manner.

### EXAMPLES

The following embodiments demonstrate the advantages of the inventions.

To investigate process effects, small scale lab batches of monolithic prototypes were manufactured by wet granulation (WG), roller compaction (RC), and direct compression (DC). To investigate polymer effects, a matrix of different viscosity grades (K100LV and K4M) and levels of hydroxypropyl methylcellulose (20 and 25% HPMC) was evaluated. See Table 1 for the premix preparations used in the specific Examples below.

TABLE 1
Premix preparations used in the examples
Premix Batch A: 20% HPMC (100:0 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 67.26 | 600.00 |
| MCC, NF (Avicel pH 102) | 12.56 | 112.00 |
| HPMC, USP K100LV Premium CR | 20.18 | 180.00 |
| HPMC, USP K4M Premium CR | 0.00 | 0.00 |
| TOTAL | 100.00 | 892.00 |

Premix Batch B: 20% HPMC (67:33 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 67.26 | 600.00 |
| MCC, NF (Avicel pH 102) | 12.56 | 112.00 |
| HPMC, USP K100LV Premium CR | 13.45 | 120.00 |
| HPMC, USP K4M Premium CR | 6.73 | 60.00 |
| TOTAL | 100.00 | 892.00 |

Premix Batch C: 20% HPMC (33:67 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 67.26 | 600.00 |
| MCC, NF (Avicel pH 102) | 12.56 | 112.00 |
| HPMC, USP K100LV Premium CR | 6.73 | 60.00 |
| HPMC, USP K4M Premium CR | 13.45 | 120.00 |
| TOTAL | 100.00 | 892.00 |

Premix Batch D: 20% HPMC (0:100 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 67.26 | 600.00 |
| MCC, NF (Avicel pH 102) | 12.56 | 112.00 |
| HPMC, USP K100LV Premium CR | 0.00 | 0.00 |
| HPMC, USP K4M Premium CR | 20.18 | 180 |
| TOTAL | 100.00 | 892.00 |

Premix Batch E: 25% HPMC (100:0 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 63.06 | 600.00 |
| MCC, NF (Avicel pH 102) | 11.72 | 111.50 |
| HPMC, USP K100LV Premium CR | 25.22 | 240.00 |
| HPMC, USP K4M Premium CR | 0.00 | 0.00 |
| TOTAL | 100.00 | 951.50 |

Premix Batch F: 25% HPMC (67:33 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 63.06 | 600.00 |
| MCC, NF (Avicel pH 102) | 11.72 | 111.50 |
| HPMC, USP K100LV Premium CR | 16.82 | 160.00 |
| HPMC, USP K4M Premium CR | 8.41 | 80.00 |
| TOTAL | 100.00 | 951.50 |

Premix Batch G: 25% HPMC (33:67 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 63.06 | 600.00 |
| MCC, NF (Avicel pH 102) | 11.72 | 111.50 |
| HPMC, USP K100LV Premium CR | 8.41 | 80.00 |
| HPMC, USP K4M Premium CR | 16.82 | 160.00 |
| TOTAL | 100.00 | 951.50 |

Premix Batch H: 25% HPMC (0:100 K100LV:K4M)

| Batch Size | 4 | Kilograms |
|---|---|---|
| API Dose | 600.00 | mgs |
| Ingredient | w/w % | mg/dose |
| Ibuprofen USP 90 Grade (BASF) | 63.06 | 600.00 |
| MCC, NF (Avicel pH 102) | 11.72 | 111.50 |
| HPMC, USP K100LV Premium CR | 0.00 | 0.00 |
| HPMC, USP K4M Premium CR | 25.22 | 240 |
| TOTAL | 100.00 | 951.50 |

The following compositions were then formulated. The premixes A-H (in Table I) were blended and portions of each premix were distributed to the three manufacturing processes. The direct compression premix was blended with silicon dioxide and stearic acid and compressed (as described below). The roller compaction premixes were granulated and milled on lab scale equipment and then blended with the extragranular silicon dioxide and stearic and compressed. The wet granulation premixes were granulated, dried and milled on lab scale equipment and then blended with the extragranular silicon dioxide and stearic and compressed.

A hydrophilic matrix, controlled-release system is a dynamic one involving polymer wetting, polymer hydration, gel formation, swelling, and polymer dissolution. At the same time, other soluble excipients or drugs will also wet, dissolve, and diffuse out of the matrix while insoluble materials will be held in place until the surrounding polymer/excipient/drug complex erodes or dissolves away. The mechanisms by which drug release is controlled in matrix tablets are dependent on many variables. The main principle is that the water-soluble polymer, present throughout the tablet, hydrates on the outer tablet surface to form a gel layer. Throughout the life of the ingested tablet, the rate of drug release is determined by diffusion (if soluble) through the gel and by the rate of tablet erosion. However, contrary to this corollary of formulation science, the inventors did not use water soluble binders in the disclosed formulations. As stated above, the use of insoluble MCC allowed for an optimized release profile in the ER layer of an IR/ER ibuprofen bilayer tablet.

### Example 1

### Direct Compression Batch A

| Batch Size | 2 | Kilograms |
|---|---|---|
| API Dose | 600.00 | mgs |
| Ingredient | w/w % | mg/dose |
| Ibuprofen Pre-Mix Blend A | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 2

### Direct Compression Batch B

| Batch Size | 2 | Kilograms |
|---|---|---|
| API Dose | 600.00 | mgs |
| Ingredient | w/w % | mg/dose |
| Ibuprofen Pre-Mix Blend B | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 3

### Direct Compression Batch C

| Batch Size | 2 | Kilograms |
|---|---|---|
| API Dose | 600.00 | mgs |
| Ingredient | w/w % | mg/dose |
| Ibuprofen Pre-Mix Blend C | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 4

### Direct Compression Batch D

| Batch Size | 2 | Kilograms |
|---|---|---|
| API Dose | 600.00 | mgs |
| Ingredient | w/w % | mg/dose |
| Ibuprofen Pre-Mix Blend D | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 5

### Direct Compression Batch E

| Batch Size | 2 | Kilograms |
|---|---|---|
| API Dose | 600.00 | mgs |
| Ingredient | w/w % | mg/dose |
| Ibuprofen Pre-Mix Blend E | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 6

### Direct Compression Batch F

| Batch Size | 2 | Kilograms |
|---|---|---|
| API Dose | 600.00 | mgs |
| Ingredient | w/w % | mg/dose |
| Ibuprofen Pre-Mix Blend F | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 7

### Direct Compression Batch G

| Batch Size | 2 | Kilograms |
|---|---|---|
| API Dose | 600.00 | mgs |
| Ingredient | w/w % | mg/dose |
| Ibuprofen Pre-Mix Blend G | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 8

### Direct Compression Batch H

| Batch Size | 2 | Kilograms |
|---|---|---|
| API Dose | 600.00 | mgs |
| Ingredient | w/w % | mg/dose |
| Ibuprofen Pre-Mix Blend H | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 9

### Roller Compaction Batch A

| Batch Size | 0.9 | Kilograms |
|---|---|---|
| API Dose | 600.00 | mgs |
| Ingredient | w/w % | mg/dose |
| Ibuprofen Milled RC A | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 10

### Roller Compaction Batch B

| Batch Size | 0.9 | Kilograms |
|---|---|---|
| API Dose | 600.00 | mgs |
| Ingredient | w/w % | mg/dose |
| Ibuprofen Milled RC B | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 11

### Roller Compaction Batch C

| Batch Size | 0.9 | Kilograms |
|---|---|---|
| API Dose | 600.00 | mgs |
| Ingredient | w/w % | mg/dose |
| Ibuprofen Milled RC C | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 12

### Roller Compaction Batch D

| Batch Size | 0.9 | Kilograms |
|---|---|---|
| API Dose | 600.00 | mgs |
| Ingredient | w/w % | mg/dose |
| Ibuprofen Milled RC D | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 13

### Roller Compaction Batch E

| Batch Size | 0.9 | Kilograms |
|---|---|---|
| API Dose | 600.00 | mgs |
| Ingredient | w/w % | mg/dose |
| Ibuprofen Milled RC E | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 14

### Roller Compaction Batch F

| Batch Size | 0.9 | Kilograms |
|---|---|---|
| API Dose | 600.00 | mgs |
| Ingredient | w/w % | mg/dose |
| Ibuprofen Milled RC F | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 15

### Roller Compaction Batch G

| Batch Size | 0.9 | Kilograms |
|---|---|---|
| API Dose | 600.00 | mgs |
| Ingredient | w/w % | mg/dose |
| Ibuprofen Milled RC G | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 16

### Roller Compaction Batch H

| Batch Size | 0.9 | Kilograms |
|---|---|---|
| API Dose | 600.00 | mgs |
| Ingredient | w/w % | mg/dose |
| Ibuprofen Milled RC H | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 17

### Wet Granulation Batch A

| Batch Size | 1 | Kilograms |
|---|---|---|
| API Dose | 600.00 | mgs |
| Ingredient | w/w % | mg/dose |
| Ibuprofen Milled WG A | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 18

### Wet Granulation Batch B

| Batch Size | 1 | Kilograms |
|---|---|---|
| API Dose | 600.00 | mgs |
| Ingredient | w/w % | mg/dose |
| Ibuprofen Milled WG B | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 19

### Wet Granulation Batch C

| Batch Size | 1 | Kilograms |
|---|---|---|
| API Dose | 600.00 | mgs |
| Ingredient | w/w % | mg/dose |
| Ibuprofen Milled WG C | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 20

### Wet Granulation Batch D

| Batch Size | 1 | Kilograms |
|---|---|---|
| API Dose | 600.00 | mgs |
| Ingredient | w/w % | mg/dose |
| Ibuprofen Milled WG D | 98.67 | 892.00 |
| Silicon Dioxide Colloidal NF Aerosil 200 | 0.88 | 8.00 |
| Stearic Acid, NF Powder Food Grade | 0.44 | 4.00 |
| TOTAL | 100.00 | 904.00 |

### Example 21

### Wet Granulation Batch E

| Batch Size | 1 | Kilograms |
|---|---|---|
| API Dose | 600.00 | mgs |
| Ingredient | w/w % | mg/dose |
| Ibuprofen Milled WG E | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 22

### Wet Granulation Batch F

| Batch Size | 1 | Kilograms |
|---|---|---|
| API Dose | 600.00 | mgs |
| Ingredient | w/w % | mg/dose |
| Ibuprofen Milled WG F | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 23

### Wet Granulation Batch G

| Batch Size | 1 | Kilograms |
|---|---|---|
| API Dose | 600.00 | mgs |
| Ingredient | w/w % | mg/dose |
| Ibuprofen Milled WG G | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

### Example 24

### Wet Granulation Batch H

| Batch Size | 1 | Kilograms |
|---|---|---|
| API Dose | 600.00 | mgs |
| Ingredient | w/w % | mg/dose |
| Ibuprofen Milled WG H | 98.67 | 951.50 |
| Silicon Dioxide Colloidal NF Aerosil 200 | 0.88 | 8.53 |
| Stearic Acid, NF Powder Food Grade | 0.44 | 4.27 |
| TOTAL | 100.00 | 964.30 |

Viscosity* of the specific Examples was determined and summarized in Table 2 below.
* Viscosity of a 2% w/w HPMC solution in water, cps

TABLE 2
Viscosity Results for HPMC Examples

| | | | | | 2% w/w |
|---|---|---|---|---|---|
| Examples | K100LV | wt % | K4M | Wt % | SoIn Visc |
| 1, 5, 9, 13, 17, 21 | 100 | 1 | 4000 | 0 | 100 |
| 2, 6, 10, 14, 18, 22 | 100 | 0.67 | 4000 | 0.33 | 411 |
| 3, 7, 11, 15, 19, 23 | 100 | 0.33 | 4000 | 0.67 | 1414 |
| 4, 8, 12, 16, 20, 24 | 100 | 0 | 4000 | 1 | 4000 |

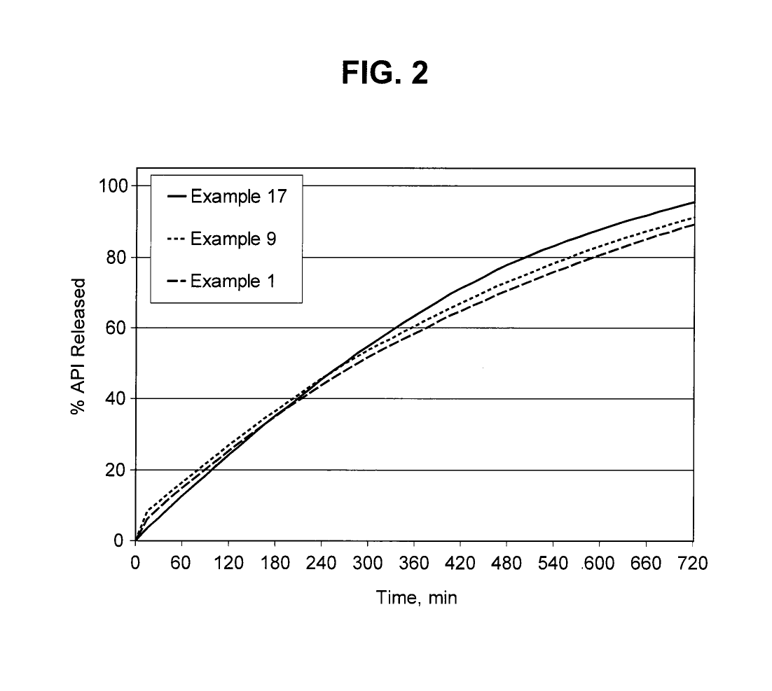

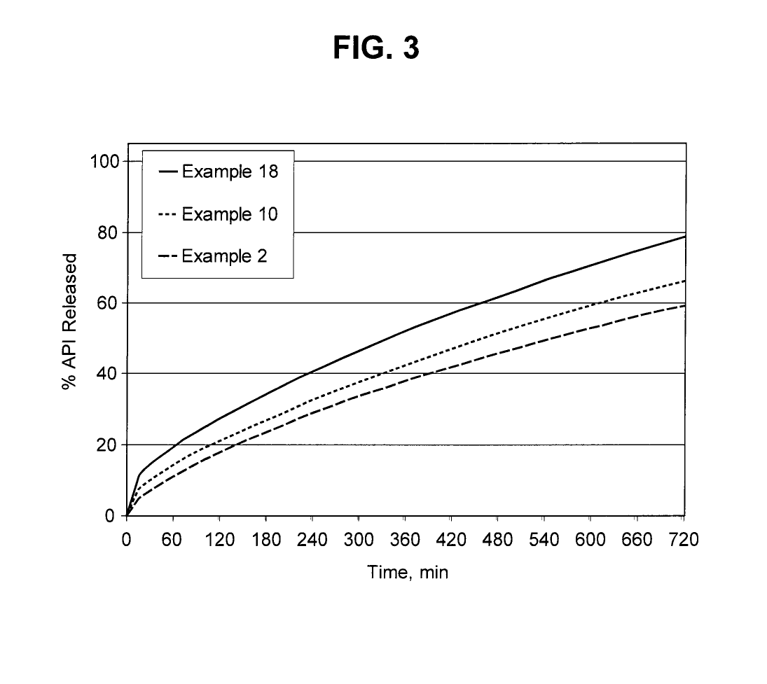

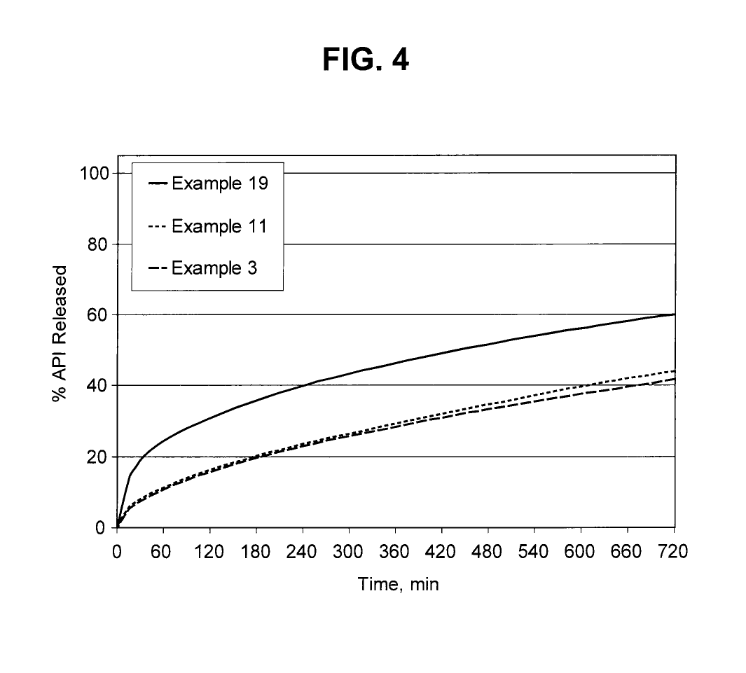

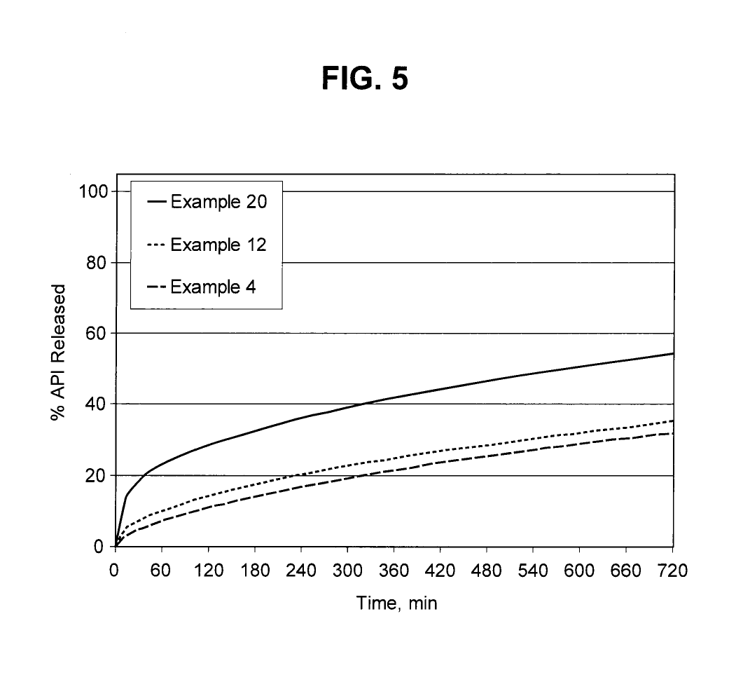

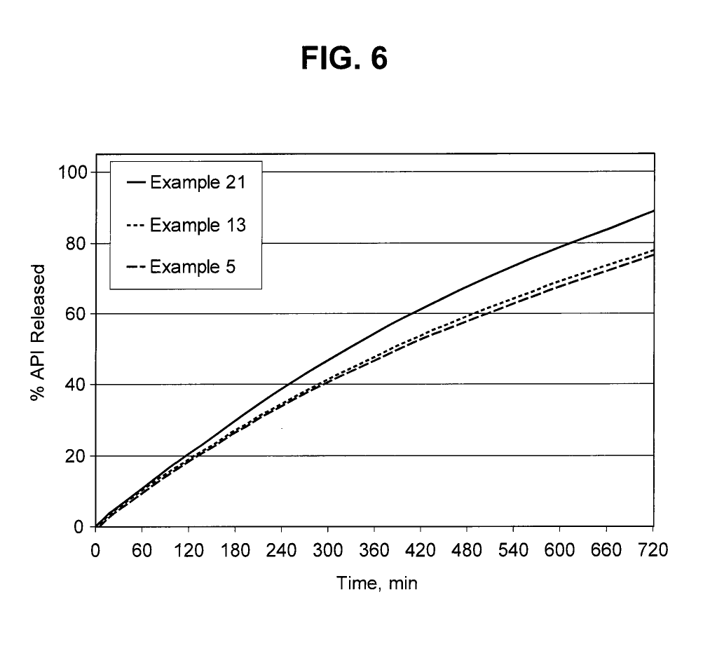

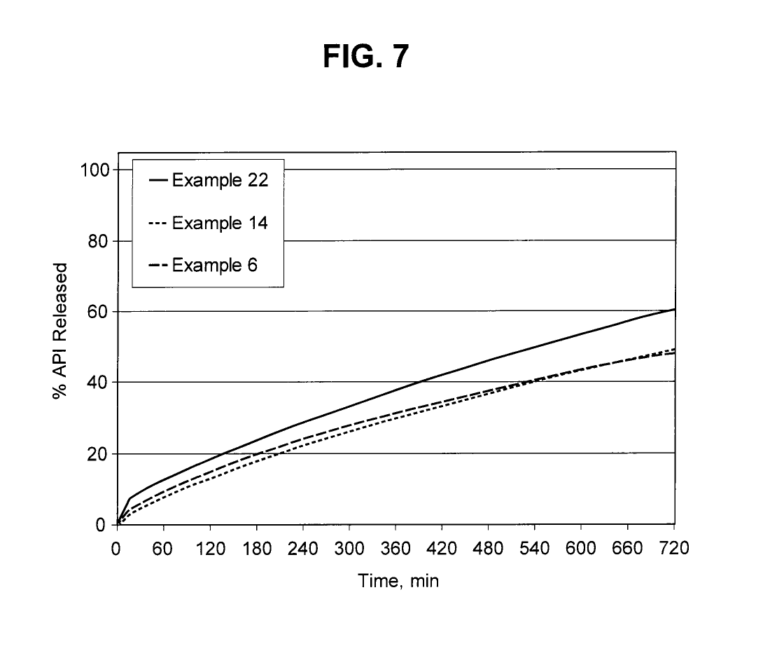

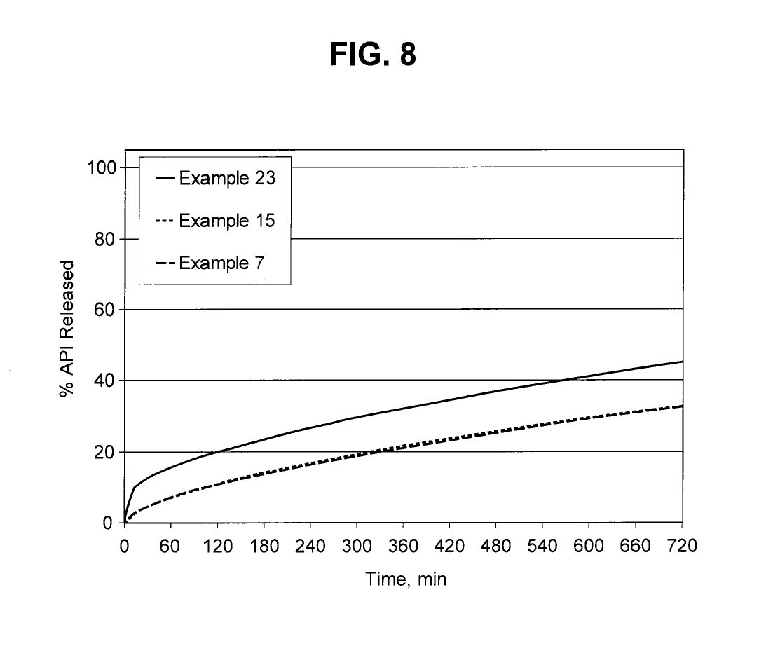

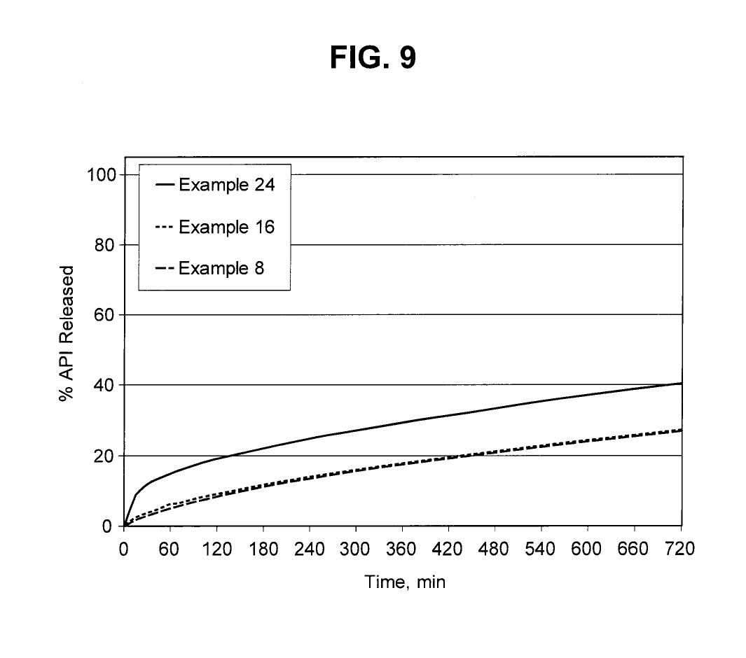

The amount of burst release was expressed as a ratio as shown below, where the denominator is calculated from the linear release rate in the region from 60 to 720 mins. FIGS. 2-9 show the profiles and comparisons of wet granulation, roller compaction and direct compression processed compositions.

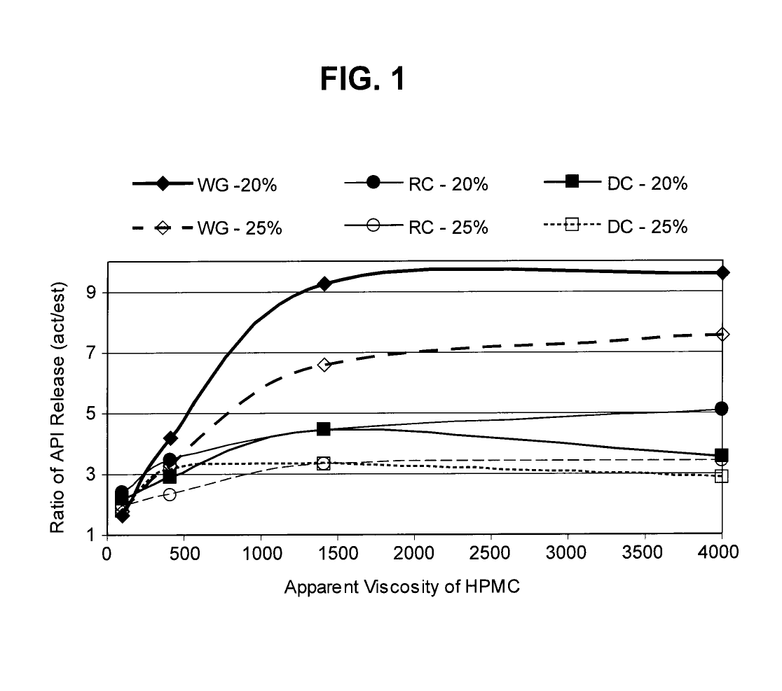

FIG. 1 summarizes the comparison API Release and composition viscosity in compositions processed with wet granulation, roller compaction and direct compression processes.

Results:

Higher polymer levels were associated with lower release rates and lower levels of burst drug release. As the polymer level increases, the API release rate and burst rate decreases as this creates a more robust gel matrix.

Polymer viscosity also has a strong correlation with burst levels. Increasing the proportion of higher viscosity polymer increases the amount of burst release, which is a function the hydration rates of the HPMC. This reduced hydration level of the polymer allows release of API before the gel matrix develops.

Process factors had the most dramatic effect on burst release. Dry processes had lower levels of burst release vs. wet granulation. These results suggest that the hydration/dehydration steps of the wet granulation process increase the amount of burst release.

Conclusions:

This set of BCS Class II, high drug load prototypes showed that burst release can be minimized by using higher levels of HPMC, selecting lower viscosity polymer grades, and using dry processing methods.

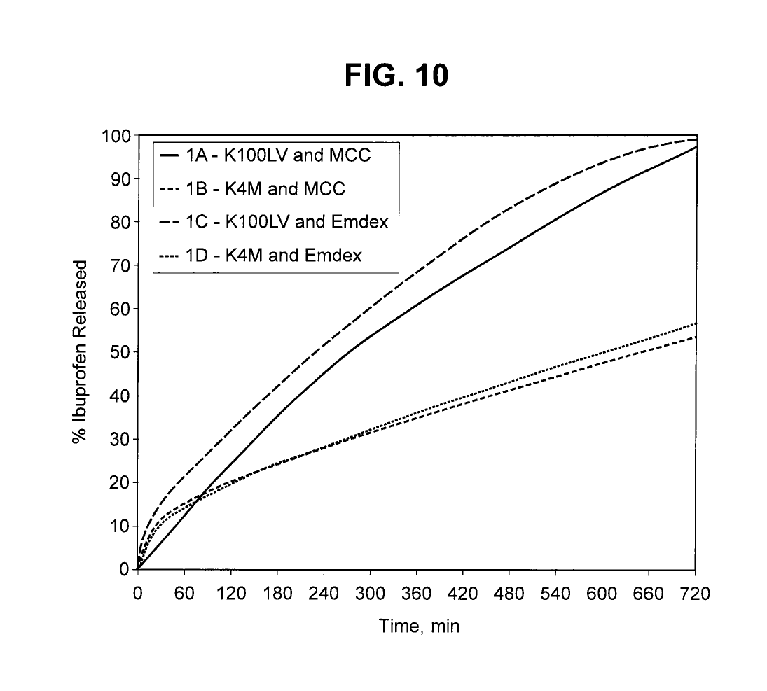

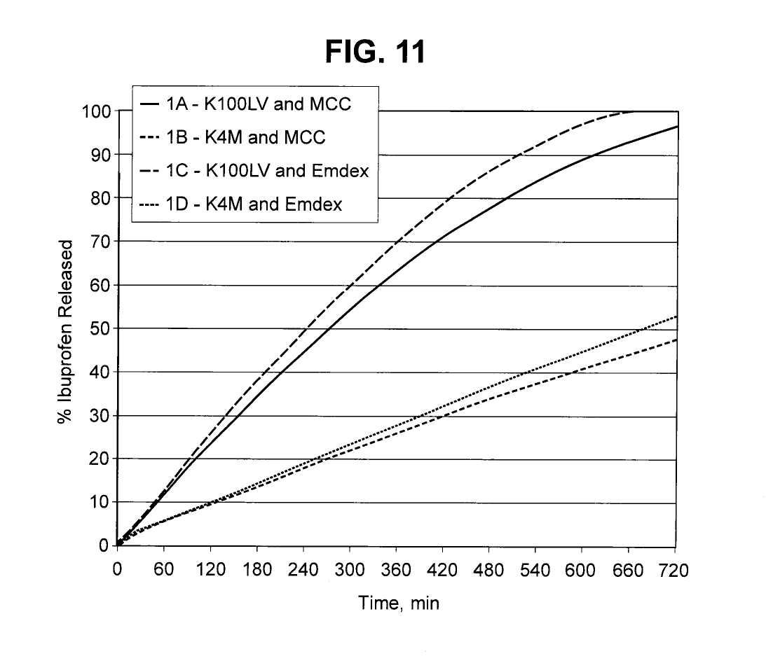

FIGS. 10 and 11 show the differences in the release profile of ibuprofen in an ER formulation when utilizing a water soluble binder (Emdex) versus an insoluble binder (MCC). Slower ibuprofen release times were shown when using insoluble binder MCC in FIG. 10 when the formulation was prepared using roller compaction. This was consistent whether the formulation included matrix forming polymers K100LV or K4M. Slower ibuprofen release times were shown when using insoluble binder MCC in FIG. 11 when the formulation was prepared using wet granulation. This too was consistent whether the formulation included K100LV or K4M.

### Example 25

This formula will be used to manufacture Ibuprofen 600 mg Caplets. This product is described as a capsule-shaped, bilayer tablet with a white film-coat. This formula is manufactured using a wet granulation method.

Formulation—Extended Release (ER) Layer

| | |
|---|---|
| 6.36 mg Stearic Acid powder | Lubricant |
| 96 mg Microcrystalline Cellulose | Low solubility Filler/Binder/Diluent |
| 400.00 mg Ibuprofen | Low solubility API |
| 6.36 mg Colloidal Silicon Dioxide | Glidant |
| 127.29 mg Hypromellose | Controlled Release Agent |

Formulation—Immediate Release (IR) Layer

| | |
|---|---|
| 1.8 mg Stearic Acid | Lubricant |
| 10.0 mg Croscarmellose Sodium | Super Disintegrant |
| 85.9 mg Corn Starch | Binder/Filler |
| 200.00 mg Ibuprofen | Low solublity API |
| 18.8 mg Pregelatinized Starch | Filler/Binder/Diluent |
| 1.6 mg Colloidal Silicon Dioxide | Glidant |
| 0.5 mg Sodium Lauryl Sulfate | Surfactant |

We claim:

1. solid dose composition comprising at least one low solubility pharmaceutically active ingredient, at least one low solubility filler and at least one controlled release agent.

2. solid dose composition of claim 1 wherein the low solubility pharmaceutically active ingredient is ibuprofen and the low solubility filler is microcrystalline cellulose.

3. method of manufacturing a solid dose composition at least one low solubility pharmaceutically active ingredient, at least one low solubility filer and at least one controlled release agent wherein at least one low solubility pharmaceutically active ingredient is premixed using wet granulation prior to tableting.

4. solid dose composition of claim 1 wherein the apparent viscosity of the controlled release agent is between 100 and 100,000 centipoise.

5. solid dose composition of claim 1 further comprising two layers wherein the layers dissolve at different rates.

6. solid dose composition of claim 5 wherein the low solubility pharmaceutically active ingredient is ibuprofen and at least one low solubility filler is microcrystalline cellulose.

7. solid dose composition of claim 5 wherein the low solubility pharmaceutically active ingredient is ibuprofen, at least one low solubility filler is microcrystalline cellulose and at least one controlled release agent is Hypromellose.

8. solid dose composition of claim 4 wherein at least one of the low solubility pharmaceutically active ingredients is ibuprofen.

9. solid dose composition of claim 4 wherein at the low solubility pharmaceutically active ingredients is ibuprofen and the low solubility filler is microcrystalline cellulose.

10. method of manufacturing a solid dose composition of claim 3 wherein at least one of the low solubility pharmaceutically active ingredients is ibuprofen.

11. solid dose composition of claim 3 wherein at least one of the controlled release agents is hydroxypropylmethylcellulose.

12. solid dose composition of claim 3 wherein at least one of the controlled release agents is hydroxypropylmethylcellulose and wherein at least one of the low solubility pharmaceutically active ingredients is ibuprofen.

13. method of manufacturing a solid dose composition of claim 2 wherein at least one of the low solubility pharmaceutically active ingredients is ibuprofen.

14. method of manufacturing a solid dose composition of claim 2 wherein at least one of the controlled release agents is hydroxypropylmethylcellulose.

15. method of manufacturing a solid dose composition of claim 2 wherein at least one of the controlled release agents is hydroxypropylmethylcellulose and wherein at least one of the low solubility pharmaceutically active ingredients is ibuprofen.

16. method of manufacturing a solid dose composition of claim 2 wherein at least one of the controlled release agents is hydroxypropylmethylcellulose, wherein at least one of the low solubility pharmaceutically active ingredients is ibuprofen and wherein at least one of the low solubility fillers is microcrystalline cellulose.
